(19) United States
(12) Patent Application Publication
(10) Pub. No.: US 2017/0340284 A1
(43) Pub. Date: Nov. 30, 2017

(54) ARTERIAL CATHETERS AND METHODS OF USE

(71) Applicant: H. LEE MOFFITT CANCER CENTER AND RESEARCH INSTITUTE, INC., Tampa, FL (US)

(72) Inventor: Tariq Chaudhry, Tampa, FL (US)

(73) Assignee: H. LEE MOFFOTT CANCER CENTER AND RESEARCH INSTITUTE, INC., Tampa, FL (US)

(21) Appl. No.: 15/522,926

(22) PCT Filed: Nov. 2, 2015
(86) PCT No.: PCT/US15/58599

§ 371 Date: Apr. 28, 2017

Related U.S. Application Data
(60) Provisional application No. 62/073,933, filed on Oct. 31, 2014.
(60) Provisional application No. 62/110,837, filed on Feb. 2, 2015.

(51) Int. Cl. A61B 5/00; A61B 5/15; A61M 25/06; A61B 5/0215

(52) U.S. Cl. CPC A61B 5/6852; A61M 25/0618; A61B 5/150992; A61B 5/0215; A61M 25/065

(57) ABSTRACT

Provided herein are systems, devices and methods for catheterization including arterial catheterization. An arterial catheter system, comprising a) a flexible tube sized for insertion into the artery of a subject defining at least one channel, b) a chamber portion connected at its distal end to the flexible tube, wherein the chamber defines an inner space that is fluidly connected to the channel of the flexible tube, c) a self-sealing membrane positioned within the inner space at the proximal end of the chamber portion so as to confine fluid within the inner space of the chamber, and d) an insertion needle having a needle point at its distal end operably connected at its proximal end to a guide wire, wherein the needle and guide wire collectively project along a longitudinal path through the self-sealing membrane into the inner space of the chamber portion and through the channel of the flexible tube.

### CROSS-REFERENCE TO RELATED APPLICATIONS

This application claims benefit of U.S. Provisional Application No. 62/073,933, filed Oct. 31, 2014, and U.S. Provisional Application Ser. No. 62/110,837, filed Feb. 2, 2015, which are hereby incorporated herein by reference in their entirety.

### BACKGROUND

Arterial catheters are placed to monitor blood pressure and to draw blood samples to check the lab values. Current arterial catheter kits have major drawbacks. Blood leakage occurs twice during the procedure increasing the cross-contamination risk. During blood draws, the dead space in the stopcock produces stagnation of blood with secondary bacterial overgrowth risking systemic infection. Absence of a needle-guard risks contaminated needle-stick injuries.

### DETAILED DESCRIPTION

Existing arterial catheter systems have a long slit in the transparent chamber to manipulate the wire from outside while maintaining the wire sterility. During the procedure, as the transparent chamber fills up with blood, it leaks out through the slit on the physician's hands promoting cross-contamination.

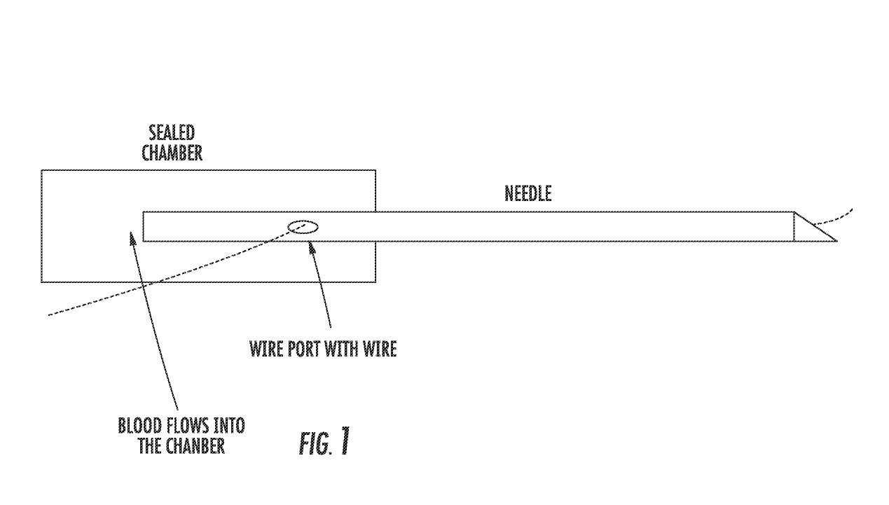

As show in FIG. 1, an example described arterial catheter is such that the transparent chamber is sealed and the needle has a side port to run the wire which is enclosed in an accordion like plastic sheath to maintain the sterility while maintaining the ability to move the wire freely. Blood from the needle tip flows into this sealed chamber and cannot leak out.

With a traditional kit, the physician is exposed to the blood once again while the system tubing is being connected to the catheter hub with the catheter tip in the artery.

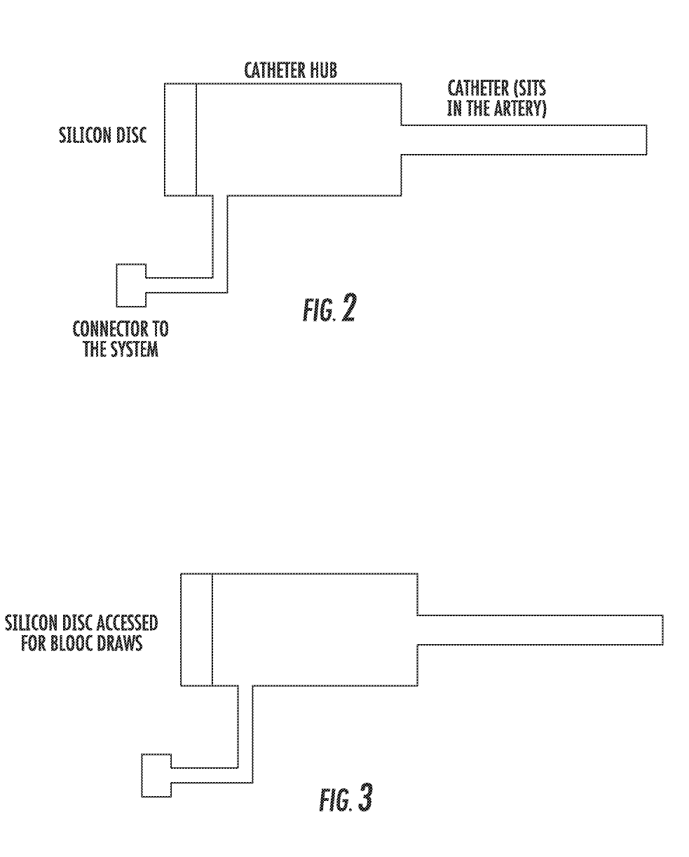

As shown in FIG. 2, a described example arterial catheter optionally has two adjustments made to the catheter. First the catheter hub has a tubing, 2-3 inches long with a connector to connect it to a monitoring system. With this modification, the catheter hub is “pre-connected” to the monitoring system eliminating the step with the above mentioned problem. Secondly, the catheter hub has a silicone disc that allows the needle through but seals shut when the needle is removed preventing blood exposure.

With a traditional kit, the stopcock is accessed to perform the blood draws. Normally the tubing is filled with saline which must be drained first for a pure blood sample. The blood drained to prime the stopcock is wasted leading to unnecessary blood loss, product waste and expensive biohazard material removal. The dead space in the stop cock also collects blood and creates a medium for bacterial growth. The syringe full of blood is deposited in the biohazard trash.

Referring to FIG. 3, the silicone disc can be accessed to perform the blood draws preventing or reducing blood waste, product waste, biohazard materials removal or dead space related infectious issues.

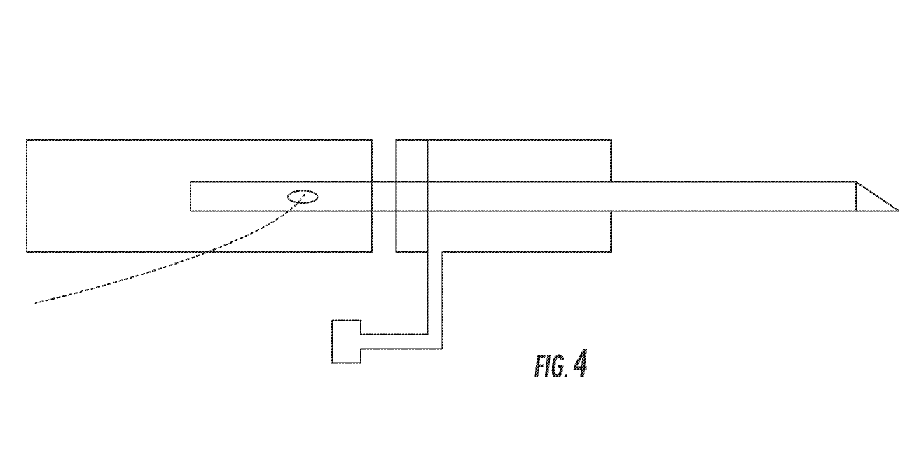

FIG. 4 shows a complete assembly of an example arterial catheter system including a sealed chamber, a needle with a wire port, a catheter, including a catheter hub with a connector for connecting to a monitoring system and a silicon disc for penetration of the needle. The system can be used a needle guard, such as described below.

Since the traditional kit does not offer a needle-guard, the needle chamber is usually dropped on the floor to prevent accidental stick by the blood contaminated needle while the physician is busy connecting the blood leaking catheter hub to the system tubing.

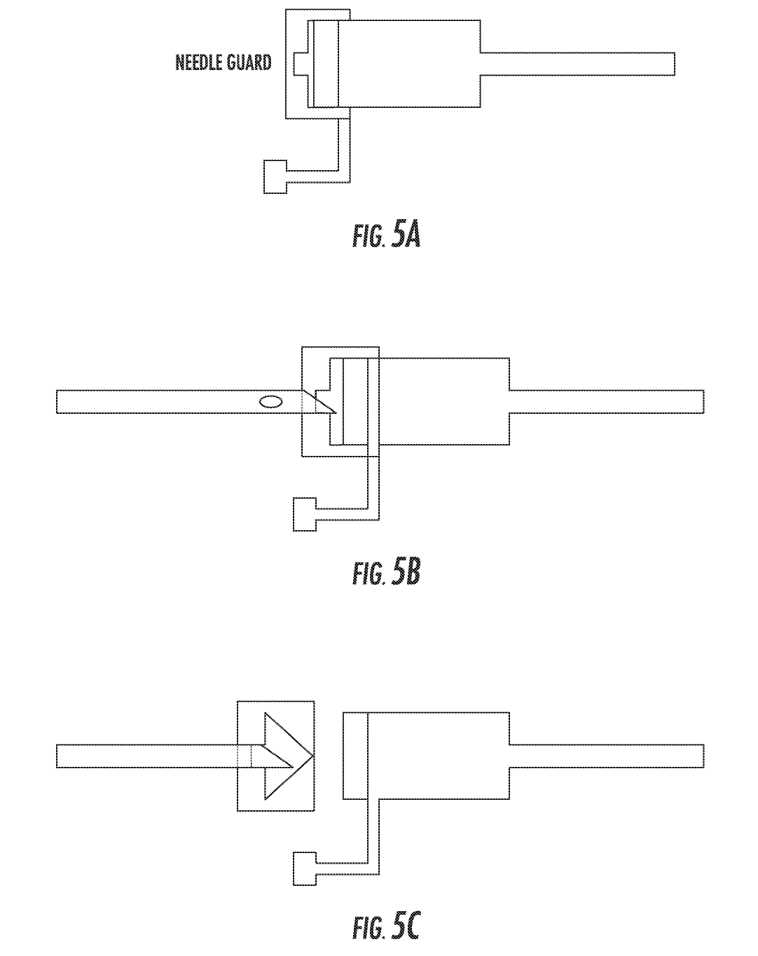

Referring to FIGS. 5A to 5C, described arterial catheters optionally include a needle guard which sits on the catheter hub during the procedure. As the needle is pulled out from the catheter at the end of the procedure, the guard grasps the needle tip and locks itself in position. When the needle is completely withdrawn, the needle-guard flaps cover the needle tip.

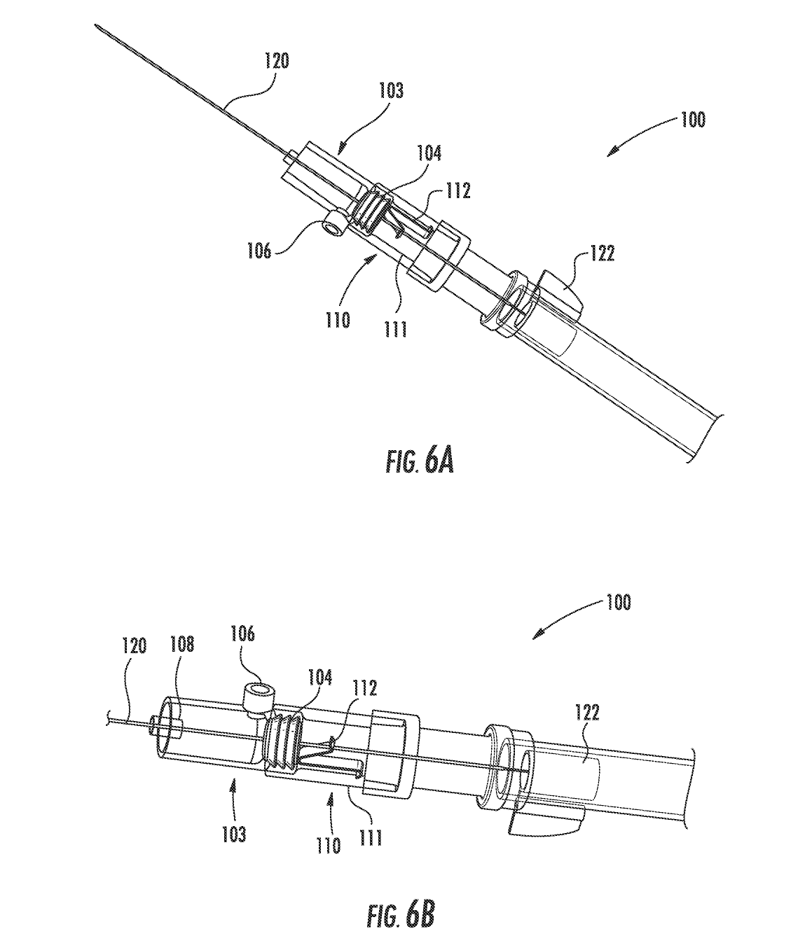

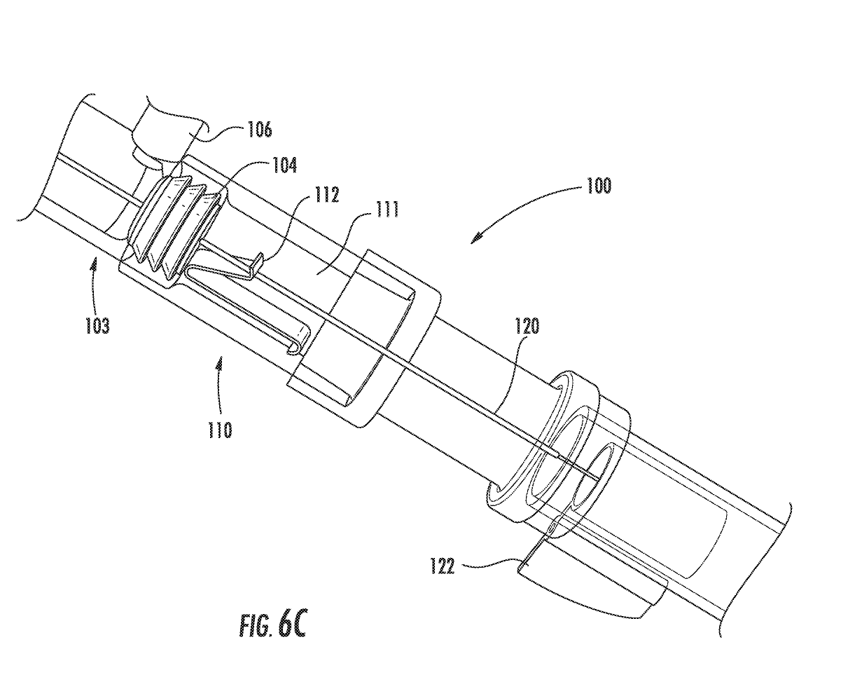

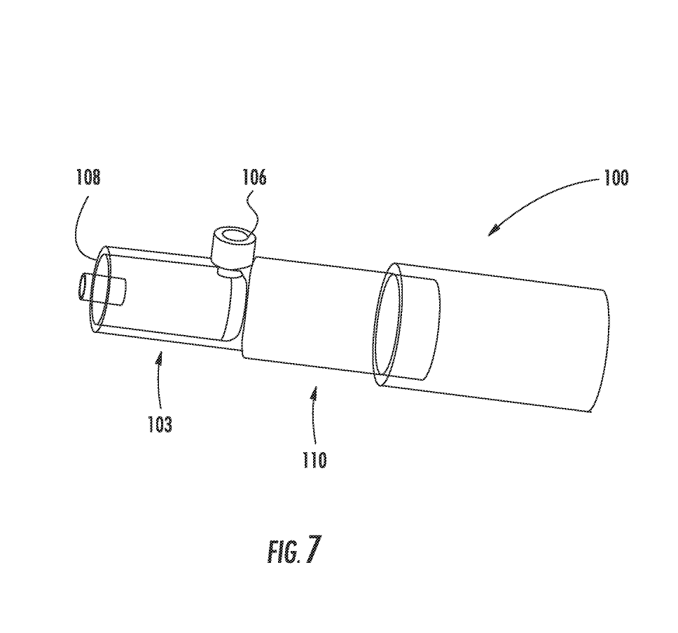

FIGS. 6A to 6C show an example described arterial access device 100 having chamber portion 103 a proximal safety portion 110 releasably connected to the proximal end of the chamber portion 103. The chamber portion 103 can be connected at its distal end to a flexible catheter tube defining at least on channel. This connection can be releasable, such as by a luer lock mechanism 108. The chamber portion 103 defines and inner space 111 that can be fluidly connected to the channel of the flexible catheter tube.

The chamber portion can further have a self-sealing membrane 104 positioned at the proximal end of the chamber portion so as to confine fluid within the inner space 111 of the chamber. As an example, the self-sealing membrane can contain a silicone material. In some embodiments, the self-sealing membrane is a plug sealingly disposed in the proximal end of the chamber.

The chamber portion 103 can further have a port 106 fluidly connected to the inner space. For example, the port 106 can be operatively connected to a pressure transducer.

The arterial access device 100 can further have an insertion needle 120 having a needle point at its distal end operably connected at its proximal end to a guide wire, wherein the needle 120 and guide wire collectively project along a longitudinal path through the self-sealing membrane 104 into the inner space 111 of the chamber portion and through the channel of the flexible tube.

The safety portion 110 contains a blocking mechanism 112 that creates a barrier preventing distal movement of the needle 120 once the distal end of the needle 120 is retracted proximally past the blocking mechanism 112. For example, the blocking mechanism 112 can involve an elastic material that is deformed while the needle 120 is advanced through the safety portion 110 so as to exert an elastic force against the needle 120 or guide wire. In these embodiments, the elastic force can move a portion of the blocking mechanism 112 into the longitudinal path of the needle 120 when the distal end of the needle 120 is retracted proximally past the blocking mechanism 112. For example, the elastic material can be spring steel.

FIG. 7 shows an example housing for the described arterial access device 100 with a chamber portion 103 having a port 106 and a luer lock mechanism 108 for connection to a catheter tube, and a safety portion 110 that is releasably connected to the chamber portion 103.

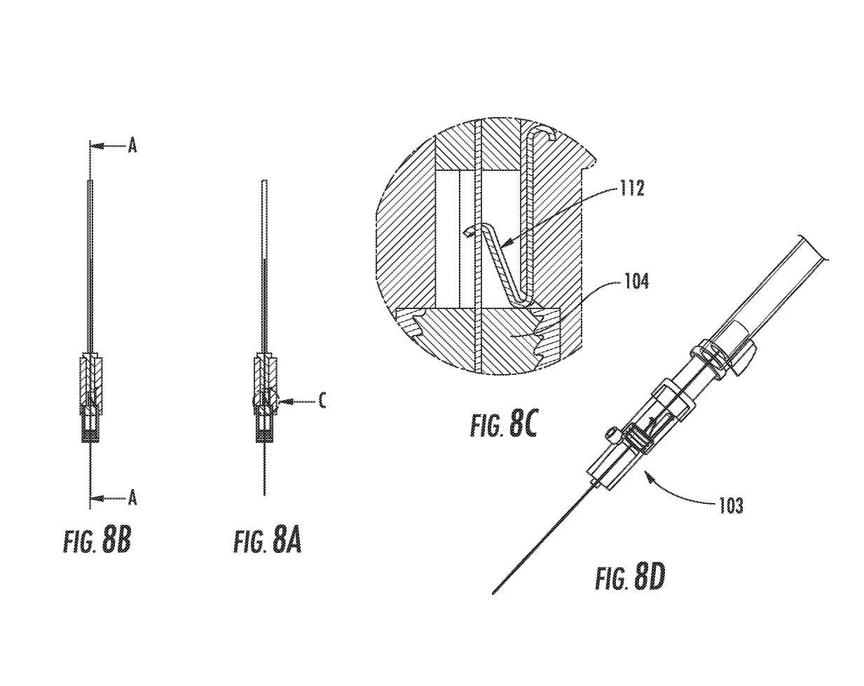

FIGS. 8A-8D show an example described arterial access device having a proximal safety portion 110 containing a blocking mechanism 112 that prevents distal advancement of the needle 120 once retracted by a guide wire control 122.

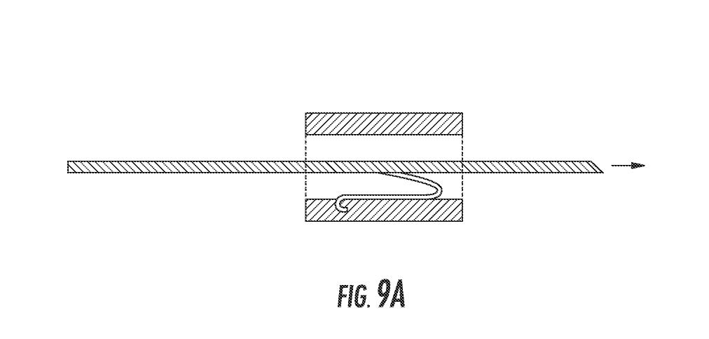

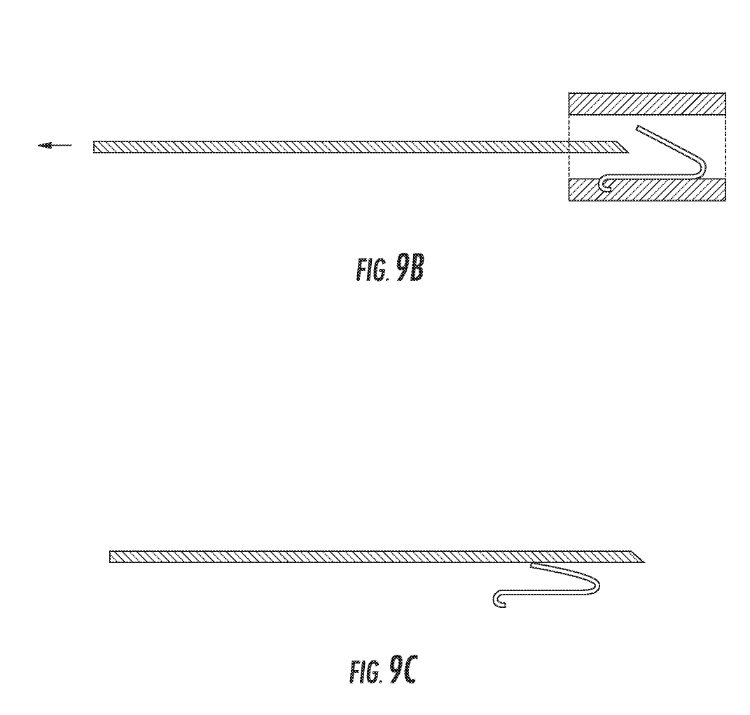

FIGS. 9A to 9D show an example blocking mechanism based on an elastically deformed member that creates a barrier for distal movement of the needle once the needle is retracted proximally past the blocking mechanism.

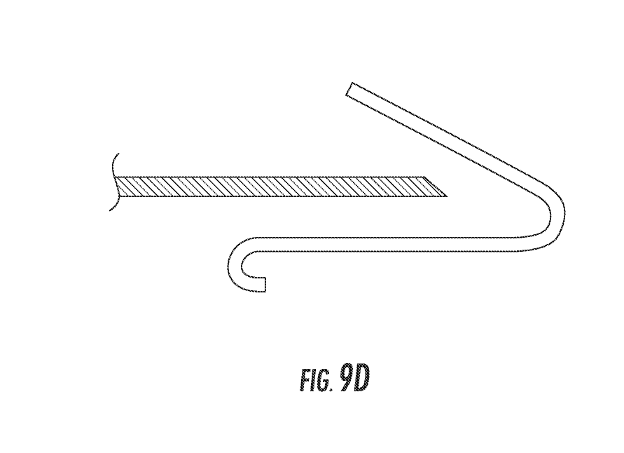

Arterial Catheters are placed to monitor constant blood pressure readings and stay in place from several hours to several days. In some cases and as early as a few hours, the plastic catheters tend to kink which is a function of the warm blood temperature and the dip catheter takes upon entering the artery. The normal waveform is lost and patient's blood pressure cannot be monitored. FIG. 10A shows normal blood pressure waveform, and FIG. 10B shows dampening of the blood pressure waveform related to catheter kinking. At times, the malfunctioning catheter needs to be replaced with a fresh catheter which takes times and utilization of the valuable hospital resources.

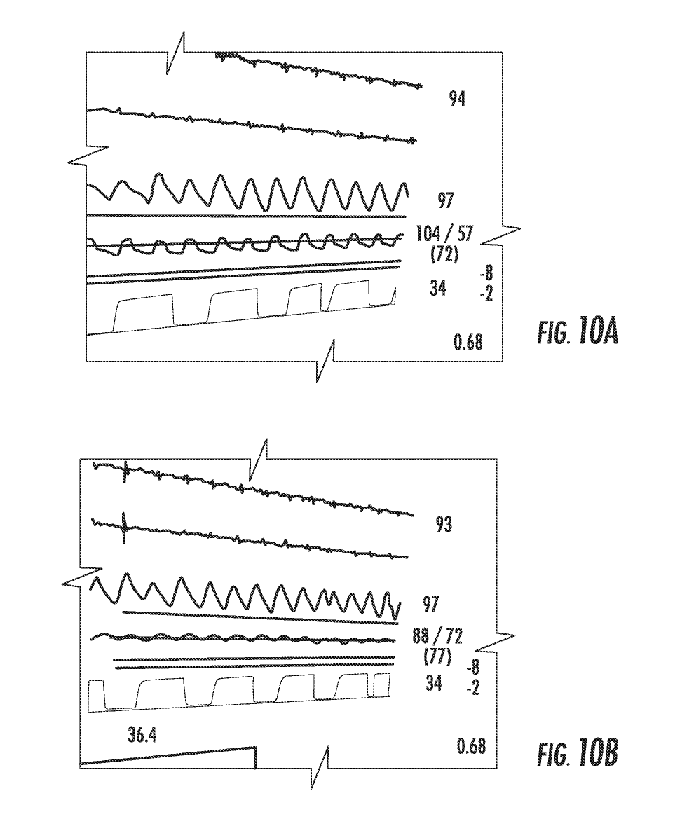

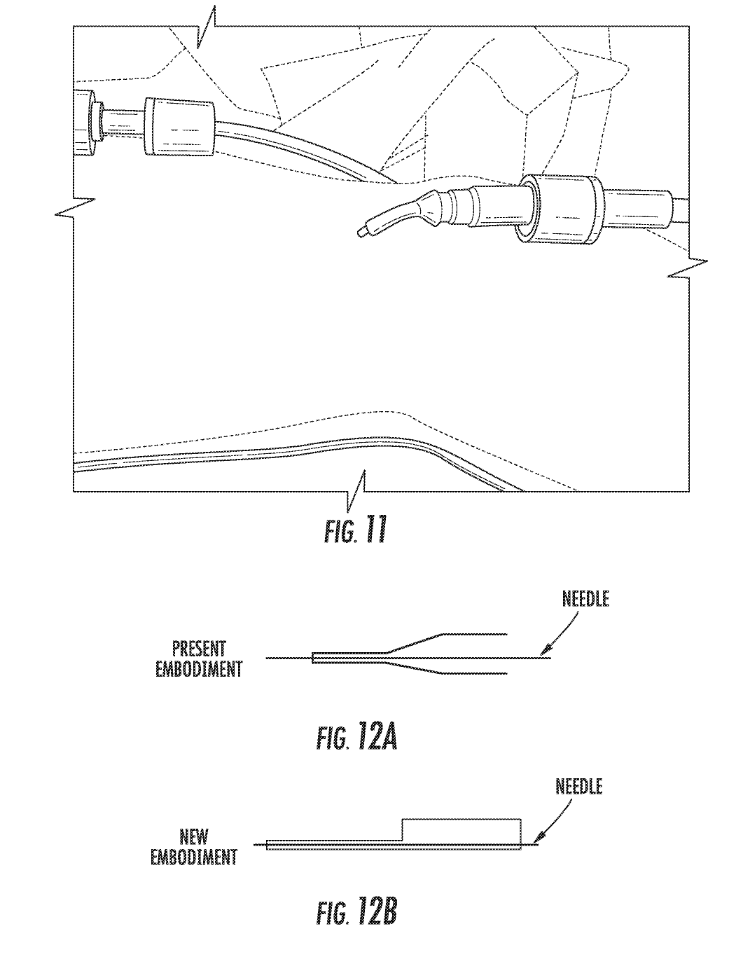

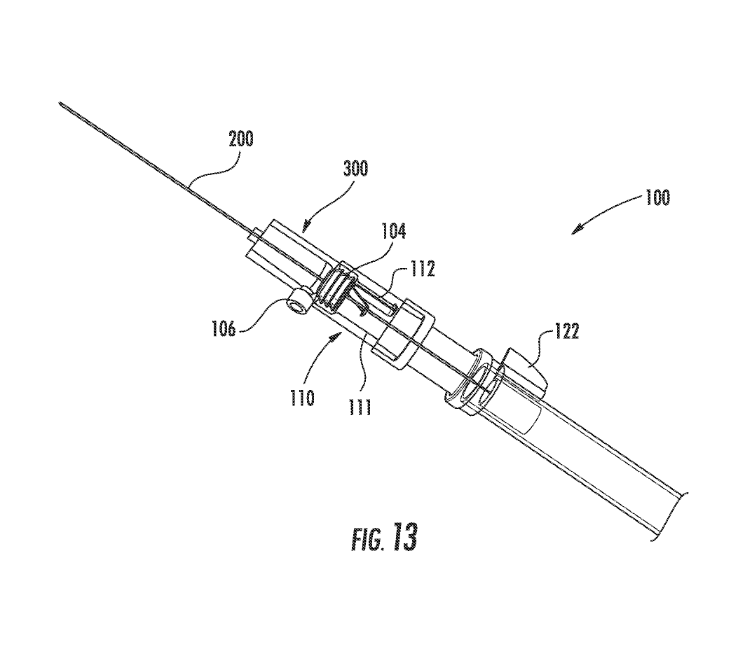

FIG. 11 shows the “dip” a normal catheter takes upon piercing the skin and entering the artery. FIG. 12A illustrates a traditional catheter where the needle passes through the center of the catheter hub, and the catheter tapers uniformly from the catheter hub. FIG. 12B illustrates an alternative conformation where the needle passage is offset away from the center of the catheter hub. In these embodiments, the catheter can extend from the catheter hub with little or no tapering along one edge. In these embodiments, the catheter can run parallel to the skin minimizing the dip and preventing catheter kink related blockages when the catheter is aligned so that the edge of the catheter hub closest to the needle is in contact with the skin. FIG. 13 shows an example described arterial access device 100 where the needle 120 and guide wire project along a longitudinal path that is offset from the longitudinal axis (center).

The disclosed arterial catheters can be inserted into any accessible artery, such as a radial artery, brachial artery, femoral artery, dorsalis pedis artery, or ulnar artery. In particular embodiments, the disclosed arterial catheters are used at sites where standard plastic catheters have the tendency to kink and malfunction over time.

A number of embodiments of the invention have been described. Nevertheless, it will be understood that various modifications may be made without departing from the spirit and scope of the invention.

Disclosed are materials, systems, devices, compositions, and components that can be used for, can be used in conjunction with, can be used in preparation for, or are products of the disclosed methods, systems and devices. These and other components are disclosed herein, and it is understood that when combinations, subsets, interactions, groups, etc. of these components are disclosed that while specific reference of each various individual and collective combinations and permutations of these components may not be explicitly disclosed, each is specifically contemplated and described herein. For example, if a method is disclosed and discussed each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed systems or devices. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

1. An arterial catheter system, comprising
a) a flexible tube sized for insertion into the artery of a subject defining at least one channel,
b) a chamber portion connected at its distal end to the flexible tube, wherein the chamber defines an inner space that is fluidly connected to the channel of the flexible tube,
c) a self-sealing membrane positioned within the inner space at the proximal end of the chamber portion so as to confine fluid within the inner space of the chamber, and
d) an insertion needle having a needle point at its distal end operably connected at its proximal end to a guide wire, wherein the needle and guide wire collectively project along a longitudinal path through the self-sealing membrane into the inner space of the chamber portion and through the channel of the flexible tube.

2. The system of claim 1, wherein the chamber portion further comprises a port fluidly connected to the inner space.

3. The system of claim 2, wherein the port is operatively connected to a pressure transducer.

4. The system of claim 1, wherein the chamber portion is releasably connected to the flexible tube by a luer lock mechanism.

5. The system of claim 1, further comprising a safety portion releasable connected to the proximal end of the chamber portion, wherein the safety portion comprises a blocking mechanism that creates a barrier preventing distal movement of the needle once the distal end of the needle is retracted proximally past the blocking mechanism.

6. The system of claim 5, wherein the blocking mechanism comprises an elastic material that is deformed while the needle is advanced through the safety portion so as to exert an elastic force against the needle or guide wire, wherein the elastic force moves a portion of the blocking mechanism into the longitudinal path when the distal end of the needle is retracted proximally past the blocking mechanism.

7. The system of claim 6, wherein the elastic material comprises spring steel.

8. The system of claim 1, wherein the self-sealing membrane comprise a silicone material.

9. The system of claim 1, wherein the self-sealing membrane is a plug sealingly disposed in the proximal end of the chamber.

10. The system of claim 1, wherein the longitudinal path of the needle and guide wire is offset from the longitudinal axis.

11. A method of accessing the vasculature of a subject, comprising inserting a portion of the catheter system of claim 1 into the vasculature of the subject.
